(19) United States
(12) Patent Application Publication
(10) Pub. No.: US 2011/0105811 A1
(43) Pub. Date: May 5, 2011

(54) PRODUCTION OF DISTILLATE BLENDING COMPONENTS

(76) Inventors: Dennis J. O'Rear, Penngrove, CA (US); Sven Ivar Hommeltoft, Pleasant Hill, CA (US); Steve S. Mathur, Danville, CA (US); Jerome F. Mayor, Lincoln, CA (US); Hye-Kyung Timken, Albany, CA (US)

(21) Appl. No.: 12/610,010

(22) Filed: Oct. 30, 2009

(51) Int. Cl. C07C 7/20; C07C 2/62; C07C 2/60

(52) U.S. Cl. 585/1; 585/317; 585/332; 585/375; 585/721; 585/727

(57) ABSTRACT

A process to produce an alkylate distillate blending component in one embodiment comprising: providing at least one olefinic C5+ product which was produced by conversion of synthesis gas in a Fischer Tropsch process; and alkylating the olefinic C5+ product in the presence of an acidic ionic liquid alkylation catalyst with hydrocarbons selected from the group consisting of isoparaffins, cycloparaffins, and their mixtures to form an alkylate distillate blending component is described.

### FIELD OF THE INVENTION

The present invention relates to the production of distillate blending components from olefinic, low-aromatic feeds such as Fischer Tropsch condensate.

### BACKGROUND OF THE INVENTION

The Fischer Tropsch process has long been used to convert hydrocarbonaceous feedstocks into salable transportation fuels.

Two general variations of the process exist, a High Temperature Fischer Tropsch (HTFT) which forms C₄− gases and C₅+ liquids that contain olefins and aromatics; and a Low Temperature Fischer Tropsch (LTFT) process which forms C₄− gases, C₅+ liquids (commonly called condensate), and at least one hydrocarbonaceous product that is solid at 20° C. (commonly called wax). The products from LTFT contain very low levels of aromatics (below 1 wt %) but may contain olefins depending on the choice of catalyst. In both LTFT and HTFT, iron catalysts yield products that contain more olefins than cobalt catalysts.

The typical salable products derived by subsequent processing of the products from the Fischer Tropsch product are condensate and salable transportation fuels (such as jet fuel and diesel fuel). The condensate is typically used for production of ethylene in olefin crackers, reforming to produce aromatic motor gasoline components, and isomerization to form isoparaffinic gasoline components. Currently there is shortage and high demand form salable transportation fuels, and the demand and price of the condensate can be less than that of salable transportation fuels. In this case there is a desire to manufacture more distillate fuel than condensate.

The current practice to manufacture salable transportation fuels from Fischer Tropsch condensates and C₄− gases is by oligomerization of olefins, typically by use of a zeolite such as ZSM-5. The products are limited to the quantity of olefins produced in the Fischer Trospch reactor and the oligomerized products are olefinic. The olefinic product must by hydrogenated to convert olefins into paraffins. Olefins create concerns over product stability.

What is desired is a process that would convert both the olefins from the C₅+ olefins from the Fischer Tropsch reactor as well as paraffins and/or cycloparaffins into blending components that can be used for salable transportation fuels. Likewise what is desired is a process that make a paraffinic product rather than an olefinic product.

Alkylation using conventional H₂SO₄ or HF acids will convert isobutane with propylene and butenes into high octane alkylate gasoline blending components. When H₂SO₄ or HF are used on C₅+ olefins they form excessive amounts of conjunct polymer, or they crack excessive amounts of heavy product back into isopentane, or the saturate the olefins by way of hydrogen transfer to form low value light paraffins. What is needed is an alkylation process which overcomes some or all of these deficiencies.

When mixtures of olefins and aromatics are alkylated with isoparaffins, the olefins alkylate directly with the aromatics without consumption of isoparaffins and form high molecular weight aromatic products that fall outside the boiling range of salable transportation fuels. Since the products from the High Temperature Fischer Tropsch product contain both olefins and aromatics, it is desirable to convert both the olefins and aromatics into alkylate distillate blending components.

### SUMMARY OF THE INVENTION

The present invention relates to a process to produce an alkylate distillate blending component in one embodiment comprising: providing at least one olefinic C₅+ product which was produced by conversion of synthesis gas in a Fischer Tropsch process; and alkylating the olefinic C₅+ product in the presence of an acidic ionic liquid alkylation catalyst with hydrocarbons selected from the group consisting of isoparaffins, cycloparaffins, and their mixtures to form an alkylate distillate blending component.

### BRIEF DESCRIPTION OF THE DRAWINGS

FIG. 1 is a process flow diagram of an embodiment of the invention wherein the Fischer Tropsch process is a Low Temperature Fischer Tropsch process.

FIG. 2 is a process flow diagram of an embodiment of the invention wherein the Fischer Tropsch process is a High Temperature Fischer Tropsch process.

### DETAILED DESCRIPTION OF THE INVENTION

### Definitions

Hydrocarbonaceous Feedstocks—Materials comprising H, C and optionally S, N, O and other elements used to manufacture synthesis gas. Examples of assets are natural gas, methane, coal, petroleum, tar sands, oils shale, shale oil, waste plastics, waste tires, municipal waste, derivatives of these and mixtures.

Synthesis gas (or syngas)—A gaseous mixture containing carbon monoxide (CO) and hydrogen and optionally other components such as water and carbon dioxide. Sulfur and nitrogen and other heteroatom impurities are not desirable since they can poison the downstream Fischer Tropsch process. These impurities can be removed by conventional techniques.

Syngas Generator: (Generation of syngas is discussed in U.S. Pat. No. 6,992,114, which is incorporated herein by reference.) This is a process or procedure to generate synthesis gas from Hydrocarbonaceous Feedstocks. A syngas generator can be a light hydrocarbon reformer using methane or natural gas as a feedstock or a heavy hydrocarbon reformer. Reforming includes a variety of technologies such as steam reforming, partial oxidation, dry reforming, series reforming, convective reforming, and autothermal reforming. All have in common the production of syngas from methane and an oxidant (steam, oxygen, carbon dioxide, air, enriched air or combinations). The gas product typically contains some carbon dioxide and steam in addition to syngas. Series reforming, convective reforming and autothermal reforming incorporate more than one syngas-forming reaction in order to better utilize the heat of reaction. The processes for producing synthesis gas from C₁-C₃ alkanes are well known to the art. Steam reformation is typically effected by contacting C₁-C₃ alkanes with steam, preferably in the presence of a reforming catalyst, at a temperature of about 1300° F. (705° C.) to about 1675° F. (913° C.) and pressures from about 10 psia (0.7 bars) to about 500 psia (34 bars). Suitable reforming catalysts which can be used include, for example, nickel, palladium, nickel-palladium alloys, and the like. Regardless of the system used to produce syngas it is desirable to remove any sulfur compounds, e.g., hydrogen sulfide and mercaptans, contained in the C₁-C₃ alkane feed. This can be affected by passing the C₁-C₃ alkane gas through a packed bed sulfur scrubber containing zinc oxide bed or another slightly basic packing material. If the amount of C₁-C₃ alkanes exceeds the capacity of the synthesis gas unit, the surplus C₁-C₃ alkanes can be used to provide energy throughout the facility. For example, excess C₁-C₃ alkanes may be burned in a steam boiler to provide the steam used in a thermal cracking step.

In a heavy hydrocarbon reformer, the process involves converting coal, heavy petroleum stocks such as resid, or combinations thereof, into syngas. The temperature in the reaction zone of the syngas generator is about 1800° F.-3000° F. and the pressure is about 1 to 250 atmospheres. The atomic ratio of free oxygen in the oxidant to carbon in the feedstock (O/C, atom/atom) is about 0.6 to 1.5, preferably about 0.80 to 1.3. The free oxygen-containing gas or oxidant may be air, oxygen-enriched air, i.e., greater than 21 up to 95 mole % O₂ or substantially pure oxygen, i.e., greater than 95 mole % O₂. The effluent gas stream leaving the partial oxidation gas generator generally has the following composition in mole % depending on the amount and composition of the feed streams: H₂: 8.0 to 60.0; CO: 8.0 to 70.0; CO₂: 1.0 to 50.0, H₂: 2.0 to 75.0, CH₄: 0.0 to 30.0, H₂S: 0.1 to 2.0, COS: 0.05 to 1.0, N₂: 0.0 to 80.0, Ar: 0.0 to 2.0. Particulate matter entrained in the effluent gas stream may comprise generally about 0.5 to 30 wt. % more, particularly about 1 to 10 wt. % of particulate carbon (basis weight of carbon in the feed to the gas generator). Fly ash particulate matter may be present along with the particulate carbon and molten slag. Conventional gas cleaning and/or purification steps may be employed such as that described in U.S. Pat. No. 5,423,894, issued Jun. 13, 1995 to Child et al.

Fischer Tropsch Process, liquid and gaseous hydrocarbons are formed by contacting a synthesis gas with a Fischer Tropsch catalyst under suitable temperature and pressure reactive conditions. The Fischer Tropsch reaction is typically conducted at temperatures of about 300 to 700° F. (149° to 371° C.), preferably about from 400° to 550° F. (204° to 228° C.); pressures of about from 10 to 600 psia, (0.7 to 41 bars), preferably 30 to 300 psia, (2 to 21 bars) and catalyst space velocities of from about 100 to about 10,000 cc/g/hr., preferably 300 to 3,000 cc/g/hr.

Fischer-Tropsch include both High Temperature: (HTFT) and Low Temperature Fischer-Tropsch (LTFT) processes, but the preferred Fischer-Tropsch process is a Low Temperature Fischer-Tropsch process, most preferably operated in a slurry bed. The HTFT processes operate at temperatures of 250° C. and above, while the LTFT process operates at below 250° C. The liquid products from the HTFT process typically contain >1 Wt % aromatics while the liquid products from the LTFT process typically contain <1 Wt % aromatics.

Ionic Liquid Alkylation Catalyst—An ionic liquid that generally in presence of a Brønsted acid component used to catalyze an alkylation reaction. Ionic liquids are liquids that are composed entirely of ions. The so-called “low temperature” Ionic liquids are generally organic salts with melting points under 100° C., often even lower than room temperature. Ionic liquids may be suitable for example for use as a catalyst and as a solvent in alkylation and polymerization reactions as well as in dimerization, oligomerization acetylation, metatheses, and copolymerization reactions.

One class of ionic liquids is fused salt compositions, which are molten at low temperature and are useful as catalysts, solvents and electrolytes. Such compositions are mixtures of components which are liquid at temperatures below the individual melting points of the components.

The most common ionic liquids are those prepared from organic-based cations and inorganic or organic anions. The most common organic cations are ammonium cations, but phosphonium and sulphonium cations are also frequently used. Ionic liquids of pyridinium and imidazolium are perhaps the most commonly used cations. Anions include, but not limited to, BF4⁻, PF₆⁻, haloaluminates such as Al₂Cl₇⁻ and Al₂Br₇⁻, [(CF₃SO₂)₂N)]⁻, alkyl sulphates (RSO₃⁻), carboxylates (RCO₂⁻) and many other. The most catalytically interesting ionic liquids for acid catalysis are those derived from heteroatom cententered cation halides such as ammonium halides and Lewis acids (such as AlCl₃, TiCl₄, SnCl₄, FeCl₃ . . . etc). Chloroaluminate ionic liquids are perhaps the most commonly used ionic liquid catalyst systems for acid-catalyzed reactions.

Examples of such low temperature ionic liquids or molten fused salts are the chloroaluminate salts. Alkyl imidazolium or pyridinium chlorides, for example, can be mixed with aluminum trichloride (AlCl₃) to form the fused chloroaluminate salts. The use of the fused salts of 1-alkylpyridinium chloride and aluminum trichloride as electrolytes is discussed in U.S. Pat. No. 4,122,245. Other patents which discuss the use of fused salts from aluminum trichloride and alkylimidazolium halides as electrolytes are U.S. Pat. Nos. 4,463,071 and 4,463,072.

U.S. Pat. No. 5,104,840 describes ionic liquids which comprise at least one alkylaluminum dihalide and at least one quaternary ammonium halide and/or at least one quaternary ammonium phosphonium halide; and their uses as solvents in catalytic reactions.

Light isoparaffins (iC₄-iC₆) can be alkylated with light olefins (C₂^{═}-C₅^{═}) using acidic ionic liquid catalysts (and in other alkylation processes with the exception of ethylene) to make clean burning alkylate gasoline.

Blending component—a hydrocarbonaceous liquid used with other blending components to make a specification transportation fuel, such as motor gasoline, aviation gasoline, jet fuel, and diesel fuel. The blending component by itself does not need to meet all the specified properties of the specification transportation fuel, but should be of us in forming the specified fuel when blended with other components. As the percentage of the blending component increases, its properties must approach those of the specification transportation fuel. Blending components are at or between 0.1 and 99.9 wt % of the specification transportation fuel, for example at or between 1 and 99%, at or between 5 and 95%, at or between 10 and 90%, at or between 20 and 80%, at or between 30 and 70%.

Specification transportation fuel: a transportation fuel of the group of motor gasoline, aviation gasoline, jet fuel, and diesel fuel that conforms to at least one of the following standards.

Diesel fuel: A material suitable for use in diesel engines and conforming to the current version of at least one of the following specifications: ASTM D-975, “Standard Specification for Diesel Fuel Oils” European Grade CEN 90 Japanese Fuel Standards JIS K 2204 The United States National Conference on Weights and Measures (NCWM) 1997 guidelines for premium diesel fuel The United States Engine Manufacturers Association recommended guideline for premium diesel fuel (FQP-1A)

Distillate fuel: A material containing hydrocarbons with boiling points between about 60 and 1100° F. The term “distillate” means that typical fuels of this type can be generated from vapor overhead streams of petroleum crude distillation. In contrast, residual fuels cannot be generated from vapor overhead streams of petroleum crude distillation, and are a non-vaporizable remaining portion. Within the broad category of distillate fuels are specific fuels that include: naphtha, jet fuel, diesel fuel, kerosene, aviation gasoline, fuel oil, and blends thereof.

Gasoline: A material suitable for use in spark-ignition internal-combustion engines for automobiles and light trucks (motor gasoline) and piston engine aircrafts (aviation gasoline) meeting the current version of at least one of the following specifications: ASTM D-4814 for motor gasoline European Standard EN 228 for motor gasoline Japanese Standard JIS K2202 for motor gasoline ASTM D-910 for aviation gasoline ASTM D-6227, “Standard Specification for Grade 82 Unleaded Aviation Gasoline” UK Ministry of Defence Standard 91-90/Issue 1 (DERD 2485), GASOLINE, AVIATION: GRADES 80/87, 100/130 and 100/130 LOW LEAD

Jet fuel: A material suitable for use in turbine engines for aircrafts or other uses meeting the current version of at least one of the following specifications: ASTM D-1655 DEF STAN 91-91/3 (DERD 2494), TURBINE FUEL, AVIATION, KEROSINE TYPE, JET A-1, NATO CODE: F-35 International Air Transportation Association (IATA) “Guidance Material for Aviation Turbine Fuels Specifications,” 4th edition, March 2000 United States Military Jet fuel specifications MIL-DTL-5624 (for JP-4 and JP-5) and MIL DTL-83133 (for JP-8)

Hydrocracking: A process that may be conducted according to conventional methods known to those of skill in the art, but with controlling the conditions for hydrocracking such that a distillate fuel product comprising a moderate amount of aromatics and the above-described properties is provided. The hydrocracking process is effected by contacting the particular fraction or combination of fractions, with hydrogen in the presence of a suitable hydrocracking catalyst at a suitable temperature. The hydrocracking process according to the present invention is conducted at temperatures in the range of from about 600 to 900° F. (316 to 482° C.), preferably at a temperature of greater than 650° F. (343 to 454° C.), more preferably at a temperature of greater than 700° F., The hydrocracking process according to the present invention is conducted at a pressure of less than or equal to 3,000 psia, preferably less than or equal to 2,500 psia. The hydrocracking process according to the present invention is conducted using space velocities based on the hydrocarbon feedstock of about 0.1 to 2.0 hr.⁻¹. The hydrocracking process according to the present invention is conducted with hydrogen being added at a rate of 1 to 20 MSCF/B (thousand standard cubic feet per barrel), preferably 2 to 10 MSCF/B.

Hydrotreating: A process for removing impurities, such as elemental sulfur, nitrogen, or oxygen or compounds containing, sulfur, nitrogen, or oxygen, from a hydrocarbonaceous product mixture. Hydrotreating also saturates olefins and aromatics that can be present in Fischer Tropsch products to form hydrotreated distillate blending components. Typical hydrotreating conditions vary over a wide range. In general, the overall LHSV (liquid hourly space velocity) is about 0.25 to 4.0, preferably about 0.5 to 2.0. The hydrogen partial pressure is greater than 200 psia, preferably ranging from about 500 psia to about 2000 psia. Hydrogen recirculation rates are typically greater than 50 SCF/Bbl, and are preferably between 1000 and 5000 SCF/Bbl. Temperatures range from about 300° F. to about 8500° F., preferably ranging from 400° F. to 750° F.

A process to saturate benzene, other light aromatics: This process uses hydrogen at super-atmospheric pressures in the presence of a catalyst to saturate at least a portion of the benzene and heavier aromatic hydrocarbons in the feedstock to form cyclohexane, methylcyclohexane and other naphthenes and combinations. Examples of this process are naphtha isomerization, hydrocracking, and hydrodewaxing as defined in U.S. Pat. No. 6,773,578, which is incorporated by reference herein.

Naphtha Isomerization: a catalytic process which converts normal paraffins to isoparaffins. The feed is usually light virgin naphtha and the catalyst with a metal or metals with hydrogen transfer activity such as platinum or palladium on an acidic refractory support material such as alumina, zeolite, tungstated or sulfated zirconia or other acidic refractory material. Octanes may be increased by over 30 numbers when normal pentane and normal hexane are isomerized. Another beneficial reaction that occurs is that any benzene in the feed is converted to cyclohexane. Although isomerization produces high quality blend stocks, it is also used to produce feeds for alkylation and etherification processes. Normal butane, which is generally in excess in the refinery slate because of RVP concerns, can be isomerized and then converted to alkylate or to methyl tert-butyl ether (MTBE) with a small increase in octane and a large decrease in RVP.

The present invention uses at least one olefinic C₅+ product which in one embodiment was produced by conversion of synthesis gas in a Fischer Tropsch process in an alkylation process with hydrocarbons selected from the group consisting of isoparaffins, cycloparaffins and their mixtures to form an alkylate distillate blending component. The isoparaffins and cycloparaffins may also be derived from a Fischer Tropsch process and in an embodiment are produced from the same Fischer Tropsch process from which the at least one olefinic C₅+ product was produced.

In an embodiment, the feedstock to the present process olefins are present >10% or >25%, ° r>50% by weight. Aromatics are present in an embodiment at concentrations low enough to avoid aromatic-olefinic alkylation in one embodiment <10%, or <2%, or <0.5% by weight In another embodiment, the feed contains low levels of branched olefins, <50% of the olefins, or <25%, or <10%, or <2%. In an embodiment, feeds having low levels of sulfur and nitrogen are employed with concentrations of <25 ppm each, or <2 ppm each, or less than 1 ppm each. FT condensate is an example. Other examples are olefinic C₅+ hydrocarbons derived from thermal cracking wax. In an embodiment the feedstock may be any refinery hydrocarbon stream which contains olefins including but not limited to streams from fluid catalytic cracking, steam cracking, thermal cracking or the Paragon Process (U.S. Pat. Nos. 4,171,257; 4,251,348; 4,282,085; 4,390,413; 4,502,945; “The Paragon Process: A New Hydrocracking Concept”, DJO'Rear, Ind. Eng. Chem. Res. 1987, 26 2337-2344). Other examples include FCC offgas, coker gas, olefin metathesis unit offgas, polyolefin gasoline unit offgas, methanol to olefin unit offgas and methyl-t-butyl ether unit offgas.

Since the catalysts used in the alkylation reaction stage will alkylate cycloparaffins with olefins, in an embodiment, the aromatics in the liquid Fischer Tropsch products are hydrogenated to form cycloparaffins such as cyclohexane, methylcyclopentane and methylcyclohexane. Olefins in this stream are hydrogenated to form paraffins. Processes to hydrogenate this liquids include hydrocracking (such as the 2^{nd} stage of a two-stage unit), naphtha hydroisomerization (such as performed with a Pt/Zeolite or Pt/halogenated alumina catalyst) and wax hydroisomerization. These processes use acidic catalyst components which also isomerize paraffins in the liquids to form isoparaffins, and isomerize cycloparaffins to form methylcycloparaffins. In an embodiment, the isoparaffins and methylcycloparaffins are alkylated with olefins using an ionic liquid catalyst to form distillate blending components and high octane alkylate gasoline blending components with little formation of light paraffins and little hydrogenation of the olefins to form paraffins.

The products from a Low Temperature Fischer Tropsch process include wax. Hydrocracking can convert this wax into isobutene, a C₅+ product that contains isoparaffins and optionally cycloparaffins, and a hydrocracked distillate blending component. Since there are few if any aromatics in the products from a Low Temperature Fischer Tropsch process, there is no need to separate C₅ olefins and alkylate them separately from heavier products. However, hydrocarbons above about C₈ can be converted directly into a hydrocracked distillate blending component by processing them in a hydrocracker.

The products from a High Temperature Fischer Tropsch process will contain aromatics which will interfere with alkylation and it is important to alkylate the C₅ olefins separately from heavier products. The C₅+ product can be fractionated to obtain C₅ olefins, an aromatic-containing C₆+ streams, and an aromatic-containing C₈+ stream. The aromatic-containing C₆+ streams can be saturated in a naphtha isomerization unit to form a mixture of isoparaffins and cycloparaffins. These can be alkylated in the alkylation unit. The aromatics in the aromatic-containing C₈+ stream can by saturated in a hydrotreater to form a hydrotreated distillate blending component.

Depending on the details of the operation, either Fischer Tropsch product may form oxygenates in the products along with the olefins. Oxygenates can deactivate alkylation catalysts and it is important to remove them by processes which minimize the saturation of the olefins. An example of a process to selectively remove oxygenates is described in US 20030158456.

Also depending on the details of the operation, either Fischer Tropsch process may form ethylene in the C₁-C₂ gas product. Ethylene is typically not a major product from the Fischer Tropsch process. However this ethylene can optionally be recovered and processed in the alkylation reactor. The ethylene does not need to be recovered as pure ethylene, but can recovered as a mixture with ethane.

The present process uses an acidic ionic liquid alkylation catalyst for the alkylation stage. Ionic liquid catalysts, such as chloroaluminates can alkylate pentene with isopentane, isohexane, isoheptane, heavier isoalkanes, methylcyclopentane, methylcyclohexane and heavier branched naphthenes and combinations to form alkylate distillate blending components and high octane alkylate gasoline blending components. Unlike alkylation using conventional H₂SO₄ or HF or zeolite, excessive polymer formation, catalyst deactivation or backwards cracking reaction is reduced with ionic liquid catalyst. It is believed that the cyclohexane is isomerized into methylcyclopentane and this compounds forms the alkylate. Likewise the hydrogen transfer activity of this catalyst is very low as shown by the low conversion of olefins into paraffins.

Furthermore, these ionic liquid catalysts have the unique ability to alkylate ethylene with isoparaffins and cycloparaffins.

All components do not necessarily need to be reacted in one system. Separate reactors can be used to react, in one embodiment, low molecular weight olefins with high molecular weight iso- and cyclo-paraffins. Other configurations will be apparent to the person of ordinary skill.

In some embodiments the present process operates at temperatures which reduces or eliminates the need for refrigeration, such as 20° C. and hotter, 30° C. and hotter, 40° C. and hotter and 50° C. and hotter.

Due to the low solubility of hydrocarbons in ionic liquids, olefins-isoparaffins alkylation, like most reactions in ionic liquids is generally biphasic and takes place at the interface in the liquid state. The catalytic alkylation reaction is generally carried out in a liquid hydrocarbon phase, in a batch system, a semi-batch system or a continuous system using one reaction stage as is usual for aliphatic alkylation. The isoparaffin and olefin can be introduced separately or as a mixture. The molar ratio between the isoparaffin and the olefin is in the range 1 to 100, for example, advantageously in the range 2 to 50, preferably in the range 2 to 20. In a semi-batch system the isoparaffin is introduced first then the olefin, or a mixture of isoparaffin and olefin. Catalyst volume in the reactor is in the range of 2 vol % to 70 vol %, preferably in the range of 5 vol % to 50 vol %. Vigorous stirring is desirable to ensure good contact between the reactants and the catalyst. The reaction temperature can be in the range −40° C. to +150° C., preferably in the range −20° C. to +100° C. The pressure can be in the range from atmospheric pressure to 8000 kPa, preferably sufficient to keep the reactants in the liquid phase. Residence time of reactants in the vessel is in the range a few seconds to hours, preferably 0.5 min to 60 min. The heat generated by the reaction can be eliminated using any of the means known to the skilled person. At the reactor outlet, the hydrocarbon phase is separated from the ionic phase by decanting, then the hydrocarbons are separated by distillation and the starting isoparaffin which has not been converted is recycled to the reactor.

Typical reaction conditions may include a catalyst volume in the reactor of 5 vol % to 50 vol %, a temperature of −10° C. to 100° C., a pressure of 300 kPa to 2500 kPa, an isoparaffin to olefin molar ratio of 2 to 16 and a residence time of 1 min to 1 hour.

A catalyst system comprised of aluminum chloride and hydrogen chloride (hydrochloric acid) for catalyzing the alkylation of iso-paraffins and olefins in ionic liquids (chloroaluminate ionic liquids) is preferred. The HCl, or any Broensted acid, can be used as a co-catalyst to enhance the reaction rate. The process according to the present invention preferably employs a catalytic composition comprising at least one aluminum halide and at least one quaternary ammonium halide and/or at least one amine halohydrate. The aluminum halide which can be used in accordance with the invention is most preferably aluminum chloride.

The quaternary ammonium halides which can be used in accordance with the invention are those described in U.S. Pat. No. 5,750,455, which is incorporated by reference herein, which also teaches a method for the preparation of the catalyst.

The ionic liquid catalysts which are most preferred for the process of the present invention are N-butylpyridinium chloroaluminate (C₅H₅C₄H₉Al₂Cl₇). A metal halide may be employed as a co-catalyst to modify the catalyst activity and selectivity. Commonly used halides for such purposes include, LiCl, BeCl₂, SiCl₂, PbCl₂, CuCl, ZrCl₄, AgCl, and PbCl₂ Preferred metal halides are CuCl, AgCl, PbCl₂, LiCl, and ZrCl₄

HCl or any Broensted acid (may be/is) employed as an effective co-catalyst. The use of such co-catalysts and ionic liquid catalysts that are useful in practicing the present invention is disclosed in U.S. Published Patent Application Nos. 2003/0060359 and 2004/0077914. Other co-catalysts that may be used to enhance the catalytic activity of ionic liquid catalyst system include IVB metal compounds preferably metal halides such as TiCl₃, TiCl₄, TiBR₃, TiBR₄, ZrCl₄, ZrBr₄, HfCL₄, HfBr₄, as described by Hirschauer et al. in U.S. Pat. No. 6,028,024.

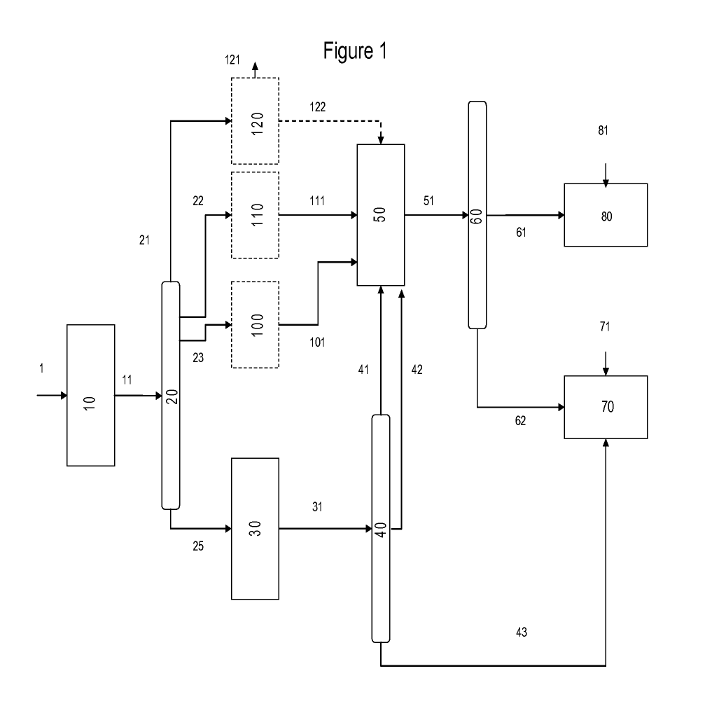

The following is a description of an embodiment of the invention with reference to FIG. 1:

Syngas (1) is converted in a Low Temperature Fischer Tropsch process (10) to form an effluent (11) which is separated in a separator (20) into a C₁-C₂ gas stream (21) which optionally contain ethylene, a C₃-C₄ stream which contains propylene and butenes and which optionally are produced as separate steams, a C₅+ olefinic stream (23), and a wax (25). The wax is hydrocracked in a hydrocracker (30) to form an effluent (31) which is separated into a stream containing isobutene (41), and a stream (42) which contains C₅+ isoparaffins and optionally cycloparaffins, an a hydrocracked distillate blending component (43). The olefins in the C₅+ olefinic stream and the C₃-C₄ stream are alkylated in an alkylation reactor (50) using an ionic liquid catalyst with the isobutane and C₅+ isoparaffins and optionally the C₅+ cycloparaffins to form and effluent (51). The effluent is distilled in a distillation unit (60) to form an alkylate gasoline blending component (61) and a alkylate distillate blending component (62). The alkylate distillate blending component is blended with the hydrocracked distillate blending component to form a blended distillate blending component (70). Optionally other distillate products (71) are blended with the blended distillate blended component. Optionally the alkylate gasoline blending component is blended with other gasoline blending components (81) to form a blended gasoline blending component (80).

Optionally a selective processes to remove oxygenates (110) is used to remove oxygenates from the C₃-C₄ stream to form a treated C₃-C₄ stream (111). Optionally a selective processes to remove oxygenates (100) is used to remove oxygenates from the C₅+ olefinic stream to form a treated C₅+ olefinic stream (101).

Optionally ethylene is recovered from the C₁-C₂ gas stream in a recovery unit (120) to form an ethylene-enriched stream (121) which is alkylated in the alkylation reactor.

Optionally a C₄-C₆ paraffinic stream (not shown) is also separated from the effluent of the alkylation unit and this C₄-C₆ paraffinic stream is isomerized in a naphtha isomerized (not shown) to form isoparaffins which are alkylated in the alkylation reactor.

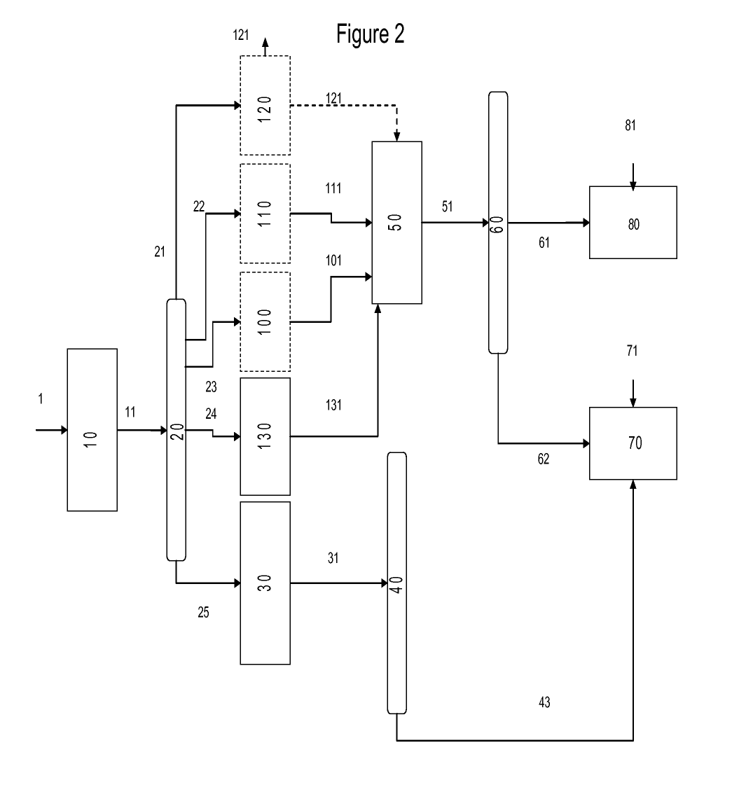

The following is a description of an embodiment of the invention with reference to FIG. 2:

Syngas (1) is converted in a High Temperature Fischer Tropsch process (10) to form an effluent (11) which is separated in a separator (20) into a C₁-C₂ gas stream (21) which optionally contain ethylene, a C₃-C₄ stream which contains propylene and butenes and which optionally are produced as separate steams, a C₅ olefinic stream (23), an aromatic-containing C₆+ stream, and an aromatic-containing C₈+ stream (25). The aromatic-containing C₈+ stream is hyrotreated in a hydrotreater (30) to form an effluent (31) which is separated into a hydrotreated distillate blending component (43). The aromatics in the aromatic-containing C₆+ stream are converted in a naphtha hydroisomerzation unit (130) to C₅+ cycloparaffins; and paraffins in this stream are converted into C₅+ isoparaffins. The olefins in the C₅ olefinic stream and the C₃-C₄ stream are alkylated in an alkylation reactor (50) using an ionic liquid catalyst with the C₅+ isoparaffins and C₅+ cycloparaffins to form and effluent (51). The effluent is distilled in a distillation unit (60) to form an alkylate gasoline blending component (61) and a alkylate distillate blending component (62). The alkylate distillate blending component is blended with the hydrotreated distillate blending component to form a blended distillate blending component (70). Optionally other distillate products (71) are blended with the blended distillate blended component. Optionally the alkylate gasoline blending component is blended with other gasoline blending components (81) to form a blended gasoline blending component (80).

Optionally a selective processes to remove oxygenates (110) is used to remove oxygenates from the C₃-C₄ stream to form a treated C₃-C₄ stream (111). Optionally a selective processes to remove oxygenates (100) is used to remove oxygenates from the C₅ olefinic stream to form a treated C₅ olefinic stream (101).

Optionally ethylene is recovered from the C₁-C₂ gas stream in a recovery unit (120) to form an ethylene-enriched stream (121) which is alkylated in the alkylation reactor.

Optionally a C₄-C₆ paraffinic stream (not shown) is also separated from the effluent of the alkylation unit and this C₄-C₆ paraffinic stream is isomerized in the naphtha hydroisomerzation unit to form isobutane and C₅+ isoparaffins which are alkylated in the alkylation reactor.

Although the present invention has been described with reference to one or more embodiments, there are numerous variations on the present invention which are possible in light of the teachings described herein. It is therefore understood that within the scope of the following claims, the invention may be practiced otherwise than as specifically described or exemplified herein.

What is claimed is:

1. A process to produce an alkylate distillate blending component comprising:
a. providing at least one olefinic C₅+ product which was produced by conversion of synthesis gas in a Fischer Tropsch process; and
b. alkylating the olefinic C₅+ product in the presence of an acidic ionic liquid alkylation catalyst with hydrocarbons selected from the group consisting of isoparaffins, cycloparaffins, and their mixtures to form an alkylate distillate blending component.

2. The process according to claim 1, wherein the acidic ionic liquid alkylation catalyst contains chloroaluminate.

3. The process according to claim 1, further comprising alkylation of olefins from a Fischer Tropsch process selected from the group consisting of ethylene, propylene, butenes and combinations.

4. The process according to claim 1, further comprising the manufacture of at least one alkylate gasoline blending component.

5. The process according to claim 1, further comprising the use of a naphtha isomerization process to produce hydrocarbons selected from the group consisting of isoparaffins, cycloparaffins, and their mixtures.

6. The process according to claim 1, wherein the Fischer Tropsch process is a Low Temperature Fischer Tropsch process and wherein the Fischer Tropsch process form a wax and wherein the wax is processed in a processes selected from the group consisting of hydrocracking and wax hydroisomeriztion to form an effluent that contains hydrocarbons selected from the group consisting of isoparaffins, cycloparaffins and their mixtures.

7. The process according to claim 6, wherein the process used to convert the wax is hydrocracking.

8. The process according to claim 7, wherein the alkylate distillate blending component is blended with the hydrocracked distillate blending component to form a blended distillate blending component.

9. The process according to claim 1, wherein the Fischer Tropsch process is a High Temperature Fischer Tropsch process and further comprising separation of the effluent from the Fischer Tropsch process into a C₅⁻ fraction which is alkylated in step (b) and an aromatic-containing C₆+ fraction.

10. The process according to claim 9, further comprising saturating at least a portion of the aromatics in the C₆+ fraction to form cycloparaffins and wherein the cycloparaffins are alkylated in step (b).

11. The process according to claim 10, wherein the process used to saturate the aromatics is selected from the group consisting of hydrocracking, hydrotreating, naphtha isomerization, and combinations thereof.

12. The process according to claim 11, further comprising the production of an aromatic-containing C₈+ fraction.

13. The process according to claim 12, further comprising saturating at least a portion of the aromatics in the C8+ fraction to form a hydrotreated distillate blending component.

14. The process according to claim 13, wherein the alkylate distillate blending component is blended with the hydrotreated distillate blending component to form a blended distillate blending component.

15. A process to produce an alkylate distillate blending component comprising:
a. providing at least one olefinic, low aromatic C₅+ containing feedstock; and
b. alkylating the feedstock in the presence of an acidic ionic liquid alkylation catalyst with hydrocarbons selected from the group consisting of isoparaffins, cycloparaffins, and their mixtures to form an alkylate distillate blending component.

16. The process according to claim 15, wherein the feedstock is selected from the group consisting of streams from fluid catalytic cracking, steam cracking, thermal cracking, the Paragon Process and their mixtures.

17. The process according to claim 15, wherein the feedstock is selected from the group consisting of FCC offgas, coker gas, olefin metathesis unit offgas, polyolefin gasoline unit offgas, methanol to olefin unit offgas, methyl-t-butyl ether unit offgas and their mixtures.

18. The process according to claim 15, wherein the paraffin stream is derived from hydrocracking or hydrotreating.
